(19) United States
(12) Patent Application Publication
(10) Pub. No.: US 2003/0125303 A1
(43) Pub. Date: Jul. 3, 2003

(54) Transdermal formulation for repair and maintenance of connective tissue

(76) Inventor: Andrew Kucharchuk, Baton Rouge, LA (US)

Correspondence Address: THOMAS S. KEATY, KEATY PROFESSIONAL LAW CORP., 2140 WORLD TRADE CENTER, NO. 2 CANAL STREET, NEW ORLEANS, LA, 70130, US

(21) Appl. No.: 10/033,930

(22) Filed: Dec. 28, 2001

(51) Int. Cl. A61K 31/7008; A61K 31/355; A61K 31/375; A61K 31/045

(52) U.S. Cl. 514/62; 514/356; 514/474; 514/729

(57) ABSTRACT

The invention describes composition and method for treatment of arthritis. The composition uses glucosamine or its salts admixed with ascorbic acid, menthol, and niacin in a cream base to form a topical agent. The method of treatment of arthritis provides for topical application of the mixed compound to the skin of the user adjacent to the inflamed or injured joint.

### BACKGROUND OF THE INVENTION

This invention relates to the use of therapeutic compositions containing glucosamine for the repair of human connective tissue, and to the transdermal formulation for treatment of the connective tissue.

Inflammatory conditions of a joint afflict and disable millions of people worldwide. A person afflicted with such chronic disease suffers from degeneration of the cartilage, the connective tissue, which cushions moveable joints. Damage to the joint cartilage is oftentimes referred as arthritis or osteoarthritis. Osteoarthritis is diagnosed when synovial joints suffer pathological changes. The cartilage and bone are slowly eroded away by reactive proliferation of bone and cartilage around the joint. Osteoarthritis is a cell-mediated active process that may result from an inappropriate response of chondrocytes to catabolic and anabolic penalytes.

The clinical manifestations include pain, tenderness, swelling and loss of function of affected joints, morning stiffness, and loss of cartilage, erosion of bone matter and subluxation of joints if the conditions are left untreated. These conditions may immobilize the patient or at least severely restrict the patient's movement.

Oftentimes, people suffering from arthritis are advised to use non-steroidal anti-inflammatory drugs (NSAID's) such as aspirin. This widely used medication, while reducing inflammation and pain associated with the degenerative cartilage condition, may adversely affect the gastrointestinal organs of the user.

In recent studies, much attention was given to amino sugar glucosamine to improve joint cartilage breakdown and arrest osteodegenerative joint disease. In some clinical studies, oral glucosamine sulfate demonstrated a potential to affect collagenase and phospholipase-A2, which is an activator of collagenase.

Glucosamine is an amino sugar and is a major constituent of hyaluronic acid; it is used in the synthesis of hyaluronic acid. Since it is essential for the normal function of a joint cartilage to have lubrication, the natural compounds causing rehydration of the cartilage will beneficially affect the joint function and the shock absorbing capability of the joint. It is believed that by increasing the amount of hyaluronic acid, glucosamine affects the hydration of the cartilage and increase in proteoglycans in the extracellular matrix of articular cartilage.

It is also believed that introduction of glucosamine into the human body leads to replenishing of the synovial fluid when cartilage is damaged due to inflammation or injury. Since glucosamine increases production of chrondroitin sulfate, it is believed to inhibit generation of degradative enzymes. Some studies indicate that the beneficial effects of glucosamine supplementation may be achieved in eight or more weeks of treatment.

Various forms of glucosamine have been tested for promoting healthy joint function in humans. One of the forms is a dietary supplement, wherein glucosamine sulfate is mixed with a protein, ginkgo-biloba and other herbs. In other studies, glucosamine sulfate was compared in results to the effects of ibuprofen taken orally for four weeks. These tests demonstrated the beneficial effect of glucosamine sulfate without the adverse effects of gastrointestinal origin.

Glucosamine may also be administered by injection, in combination with other agents, for instance collagen. However, the injection method of administering the drug suffers from the disadvantages associated with such invasive methods, requiring syringes, special training, etc.

Consequently, there exists a need for providing a medication for repairing the connective tissue in humans and for relieving effects of arthritis that may be administered through a noninvasive procedure.

### SUMMARY OF THE INVENTION

It is, therefore, an object of the present invention to provide a composition of matter suitable for use as a therapeutic agent for relieving the symptoms of an inflammatory disease of the joints, such as arthritis.

It is another object of the present invention to provide a composition of matter suitable for use as a therapeutic agent that can be easily administered and quickly metabolized in the system of the user.

It is still another object of the present invention to provide a topically applicable penetrating agent that can be easily administered by the patient.

It is a further object of the present invention to provide a method of manufacturing of a composition of matter adapted for use as a transdermal formulation for repairing the connective tissue of the joints.

These and other objects of the present invention are achieved through a provision of a composition of matter comprising therapeutically effective amounts of amino sugar glucosamine or salts thereof, mixed with a vitamin-rich cream base. The mixture is administered topically by rubbing into the skin of the user. The formulation also comprises ascorbic acid, menthol and niacin. The cream or gel base for mixing the active ingredients enhances penetration of the active ingredients through the skin of the user and helps deliver the medication to the affected area of the joint.

The cream is preferably applied at the onset of tenderness or pain in the affected joint. The transdermal introduction of glucosamine salts has a lower incidence of side affects as compared to oral preparations or injections. Addition of Vitamins C and B3 is believed to further facilitate repair of the connective joint, restoration of the joint flexibility and relieve the discomfort in the inflamed or injured joint.

### DETAILED DESCRIPTION OF THE PREFERRED EMBODIMENT

In accordance with the present invention, an effective amount of an amino sugar, or salt thereof is introduced in a transdermal formulation. The amino sugar in the preferred embodiment is glucosamine, glucosamine sulfate, or glucosamine hydrochloride. In the most preferred embodiment of the present invention, glucosamine hydrochloride is combined with ascorbic acid, niacin (vitamin B3), menthol and mixed with a penetration enhancing agent, such as pluronic-lecithin-organo (PLO) cream or gel.

Ascorbic acid or vitamin C, is a natural substance found in fruits and vegetables. Many amino acids need vitamin C to build strong cartilage. Collagen tissue, which forms the matrix of cartilage, needs vitamin C for regeneration. The ascorbic acid promotes healthy bone structure, helps in the absorption of iron, aids in the maintenance of connective tissues, such as the joint cartilage and promotes healing of wounds. Ascorbic acid is also a strong antioxidant and helps in repairing the damage done by free radicals to the connective tissue. Ascorbic acid is water soluble and is considered non-toxic when taken in moderate amounts, especially when applied topically.

Menthol and menthol containing creams are believed to increase blood circulation to the area where they are topically applied and produce a warm—or sometimes cold—feeling that is soothing and lasts for several hours. Menthol is used in topical pain relieve preparations, such as Tiger Balm, Icy-Hot, Mineral Ice, Ben-Gay and others. Menthol is used in many ointments to treat aches and pains, itching, nasal congestion and other conditions. Menthol may be produced from natural sources or synthetically.

Vitamin B3 (niacin) is a nicotinic acid that helps break down blood sugar if taken orally. It helps widen the blood vessels and increase the blood flow through the human body. Vitamin B3 can be derived from natural sources, such as salmon, mackerel, chicken, and other sources. Sometimes Vitamin B3 is prescribed for improving cholesterol levels. While high doses of vitamin B3 when taken internally, may have several side effects, such as decrease in blood pressure, liver damage, and others, topical application of the vitamin has not been shown to be of any particular danger to humans.

The PLO cream or gel of the present invention refers to pluronic-lecithin-organo compound that helps prepare the skin for administration of the active ingredients and allow for effective transport of the active ingredients into the user's joint structure. The pluronic-lecithinorgano compound of the present invention is a combination of lecithin and isopropryl palmitate. This combination is useful in pharmaceutical compounds due to the soludilizing and penetration enhancement qualities.

The term “penetration enhancement” used herein means that the materials have a direct effect on the permeability of the skin. The transdermal cream of the present invention contains lecithin isopropryl palmitate solution designed to increase percutaneous absorption of the active ingredients by the skin and ultimately into the cartilage of the affected joint.

The formulation of the present comprises between about 10% to about 12% by weight of glucosamine or a salt thereof, between about 3% to about 5% by weight of ascorbic acid, between about 1% to about 3% by weight of menthol, and between about 3% to 5% by weight of niacin, mixed with the PLO cream to make up 100% of the formulation.

To prepare the preferred embodiment of the transdermal cream used in the present invention, 100 grams of pluronic F127 20% solution was mixed with 1.5 grams of potassium sorbate NF, a preservative. Pluronic F127 and potassium sorbate NF were then thoroughly mixed with refrigerated distilled water to make up 500 ml. The mixture of powders and distilled water were then placed under refrigeration for a period of 24 hours to allow for complete dissolution of ingredients.

Lecithin isopropyl palmitate solution was prepared by mixing 200 grams of lecithin soya in a granular form with 292 ml of isopropyl palmitate NF, and 1.32 g of sorbic acid NF-FCC powder. The three ingredients were thoroughly mixed in a large beaker, covered with aluminum foil and allowed to rest for 24 hours until full dissolution of the ingredients was achieved.

Next, the carrier cream was mixed with the active ingredients. In this step of the process, for each 100 grams of the finished product, 10-12 grams of glucosamine hydrochloride, 3-5 grams of ascorbic acid, 1-3 grams of menthol, and 3-5 grams of Vitamin B3 were mixed and reduced to fine particle size by triturating with appropriate equipment. The resultant mixture was thoroughly mixed with pluronic F127 20% solution to make up 100 ml of the final product. The mixture forms a transdermal cream or gel of the present invention.

The transdermal formulation of the present invention is administered by rubbing small amounts of the cream into the skin area covering the affected joints. The daily doses can range from 25 milligrams to 500 milligrams with 50 milligrams to 100 milligrams daily dosage being preferable.

It is believed that the transdermal type of delivery of the active ingredients avoids the toxicity factor associated with oral injection of glucosamine or menthol, or injections of glucosamine. Also, the transdermal formulation of the present invention requires a lower dose of glucosamine, Vitamin C, niacin, or menthol. The user without a specific medical training may apply the medication by simply rubbing the required dose of the cream to the skin adjacent to the affected area. The PLO cream or gel helps in the process of transporting the active ingredients into the inflamed cartilage of the user.

Many changes and modifications can be made in the formulation and method of the present invention without departing from the spirit thereof. I, therefore, pray that my rights to the present invention be limited only by the scope of the appended claims.

I claim:

1. A therapeutic agent for repair of connective tissue, comprising:
an amino sugar selected from the group consisting of glucosamine sulfate and glucosamine hydrochloride adapted for topical application.

2. The therapeutic agent of claim 1, wherein the amino sugar comprises between about 10% to about 12% by total weight of resultant composition.

3. The therapeutic composition of claim 1, further comprising ascorbic acid.

4. The therapeutic agent of claim 3, wherein the ascorbic acid comprises between about 3% to about 5% by total weight of resultant composition.

5. The therapeutic composition of claim 1, further comprising menthol.

6. The therapeutic agent of claim 5, wherein the menthol comprises between about 1% to about 3% by total weight of resultant composition.

7. The therapeutic composition of claim 1, further comprising niacin.

8. The therapeutic agent of claim 7, wherein the niacin comprises between about 3% to about 5% by total weight of resultant composition.

9. The therapeutic composition of claim 1, wherein said amino sugar is mixed with a topical penetration enhancing agent.

10. The therapeutic composition of claim 9, wherein said penetration enhancing agent is pluronic-lecithin-organo cream.

11. The therapeutic composition of claim 9, wherein said penetration enhancing agent is pluronic-lecithin-organo gel.

12. A therapeutic technique for treating osteoarthritis in humans by repairing connective tissue inflammation and degeneration, comprising the step of topically administering a therapeutically effective amount of a composition comprising glucosamine mixed with a penetration enhancing agent.

13. The method of claim 12, wherein said glucosamine is glucosamine sulfate.

14. The method of claim 12, wherein said glucosamine is glucosamine hydrochloride.

15. The method of claim 12, wherein said glucosamine is mixed with ascorbic acid, menthol and niacin prior to mixing with the penetration enhancing agent.

16. The method of claim 15, wherein glucosamine is present in the amount of between about 10% to about 12% by total weight, the ascorbic acid is present in the amount of between about 3% to about 5% by total weight, menthol is present in the amount of between about 1% and 3% by total weight and niacin is present in the amount of between about 3% and about 5% by total weight.

17. A method of manufacturing a pharmaceutical composition from which effective non-toxic dosage amounts may be taken, comprising admixing:
a therapeutically effective amount of an agent for topical treatment of inflamed or damaged cartilage, said agent selected from the group consisting of glucosamine sulfate and glucosamine hydrochloride, with
a sufficient amount of topical penetration enhancing compound, said composition when manufactured comprising between about 10% to about 12% by total weight of the agent.

18. The method of claim 17, wherein said agent is admixed with pre-determined amounts of ascorbic acid, menthol and niacin prior to admixing with the penetration enhancing compound.

19. The method of claim 18, wherein said ascorbic acid is present in the manufactured composition in the amount of between about 3% to 5 by total weight, said menthol is present in the amount of between about 1% and about 3% by total weight, and said niacin is present in the amount of between about 3% and 5% by total weight of the manufactured composition.

20. A method of treating osteoarthritis in a patient comprising topical administration of an effective amount of a composition comprising glucosamine or its salts, ascorbic acid, menthol, niacin and penetration enhancing agent.

21. The method of claim 20, wherein said penetration enhancing agent is pluronic-lecithin-organo cream or gel.

22. The method of claim 21, wherein said composition comprises between about 10% to about 12% by total weight of glucosamine or its salts, between about 3% to about 5% by total weight of ascorbic acid, between about 1% and about 3% by total weight of menthol, between about 3% to about 5% by total weight of niacin, mixed with the sufficient amount of pluronic-lecithin-organo cream or gel to make 100% of the resultant composition.

23. A therapeutic composition for treating arthritis comprising an effective amount of glucosamine or its salts mixed with a topical cream.

24. The therapeutic composition of claim 23, further comprising ascorbic acid, menthol, and niacin.

25. The therapeutic composition of claim 24, comprising between about 10% to about 12% by total weight of glucosamine or its salts, between about 3% to about 5% by total weight of ascorbic acid, between about 1% and about 3% by total weight of menthol, between about 3% to about 5% by total weight of niacin, mixed with the sufficient amount of pluronic-lecithin-organo cream or gel to make 100% of the resultant composition.
